(19) United States
(12) Patent Application Publication
(10) Pub. No.: US 2002/0022008 A1
(43) Pub. Date: Feb. 21, 2002

(54) UV indicator to signal the reduction of sunscreen efficiency

(76) Inventors: Susan Ellen Forest, Cincinnati, OH (US); M. Scott Krummen, Cincinnati, OH (US); Janis Anderson Yelton, Villa Hills, KY (US)

Correspondence Address: Catherine U. Brown, The Procter & Gamble Co., Miami Valley Labs, P.O. Box 538707, Cincinnati, OH, 45253-8707, US

(21) Appl. No.: 09/885,703

(22) Filed: Jun. 20, 2001

Related U.S. Application Data
(60) Provisional application No. 60/217,426, filed on Jul. 10, 2000.

(51) Int. Cl. A61K 7/42; A61K 7/44; A61K 7/00

(52) U.S. Cl. 424/59; 424/60; 424/400; 424/401

(57) ABSTRACT

A sun protection kit and a method for preventing overexposure to ultraviolet radiation are disclosed. The sun protection kit comprises a sunscreen and an indicator that alerts a user when the sunscreen is no longer providing sufficient UV protection and needs to be reapplied. The indicator comprises a photochromic molecule, which changes color when exposed to ultraviolet radiation.

### CROSS REFERENCE

This application claims priority under Title 35, United States Code § 119(e) from Provisional Application Ser. No. 60/217,426 filed on Jul. 10, 2000.

### FIELD OF THE INVENTION

This invention relates to a sun protection kit and method for its use. More specifically, this invention relates to a sun protection kit that alerts the user when the sunscreen needs to be reapplied.

### BACKGROUND OF THE INVENTION

Ultraviolet (“UV”) radiation has wavelengths less than 400 nanometers. UV radiation is generally broken down into three major categories. UVA radiation has wavelengths in the range of 320 to 400 nanometers, UVB radiation has wavelengths in the range of 280 to 320 nanometers, and UVC covers the wavelength range less than 280 nm. UVC does not transmit through the atmosphere to the Earth's surface, however various skin physiological problems are related to exposure and over-exposure to UVA and UVB radiation in the solar spectrum. UVB was considered to be the cause of major physiological damage because it is more efficient at causing erythema than UVA. However, UVA wavelengths are more predominant in the solar spectrum than UVB, giving UVA a more important role in sun damaging effects than initially believed. While UVB is responsible for the majority of erythema (sunburn), UVA contributes to it as well. Both are suspected to be involved in photoaging of skin, which is exhibited by dryness, deep wrinkle formation, mottled pigmentation, and the breakdown of elastic tissues and collagen. While UVB is thought to be the primary wavelength region involved in skin cancer, the action spectrum for skin cancer (specifically melanoma) is not known so other regions of the solar spectrum can not be discounted as contributors as well.

One method by which consumers protect themselves against these hazards is through the use of sun protection products such as lotions, creams, sprays, or wipes. Such products are rated according to the sun protection factor, SPF. This is the ratio of the minimal erythemal dose (i.e., the UV dose required to cause minimal erythema) in protected skin as compared to the same dose in unprotected skin. It therefore describes the efficiency of a sunscreen against sunburn. Sun protection products that have a SPF>15 are generally considered “sunblocks”, while those having a SPF of 15 or less are either “sunscreen” or “suntanning” products. Many organic sunscreen and sunblock products protect the skin from UVB and UVA radiation having wavelengths up to about 365 nanometers.

For purposes of this invention, “sunscreen” means any chemical product that absorbs or scatters UV radiation and prevents UV radiation from penetrating deep into the skin (i.e., both sunblocks and sunscreens).

Without wishing to be bound by theory, it is believed that sunscreens can lose their effectiveness due to various factors. It is perceived by consumers that sunscreens can wear off by sweating, abrasion (i.e., rubbing), swimming, absorption into the skin, migration on the skin, and photodegredation, and these removal mechanisms may be important in sun protection by sunscreens. Mechanisms by which a sunscreen wears off create a problem in that a consumer cannot tell when the sunscreen has stopped providing effective UV protection.

Furthermore, consumers often fail to apply a sufficient amount of sunscreen to their skin. Commercial sunscreens typically must be applied in an amount of 2 milligrams per square centimeter to the entire region of the body that is exposed to the sun (or for an average adult, approximately 35 ml of sunscreen should be applied). The average amount actually applied by users is about 0.5 mg/cm². If a lesser amount of sunscreen is applied, then the claimed SPF value for the sunscreen is not reached and maintained. Therefore, the amount of protection that the consumer receives is related to the amount of sunscreen applied in addition to the SPF value on the bottle. Insufficient application of sunscreen will lead to lower UV protection.

Due to wear off and the initial failure to apply a sufficient amount of sunscreen, it is not possible for consumers to tell when the sunscreens are still providing adequate sun protection. Therefore, it is an object of this invention to provide a means for alerting consumers when their sunscreens are no longer protecting them from exposure to UV radiation.

One method for combating this problem is to provide a device such as a plastic gauge or monitor that can be applied to clothes or attached to skin. The device has an indicator that alerts the user when UV radiation is reaching the device. However, such gauges and devices suffer from the drawback that they are not skin-like, so the rate at which a sunscreen wears off a gauge or device does not accurately predict the status or efficiency of the sunscreen on the skin. Therefore, it is a further object of this invention to provide an indicator that will accurately predict when a sunscreen applied to the skin is still efficiently providing UV protection.

Gauges and devices known in the art also suffer from the drawback that they are not essentially UV transparent. Sunscreens do not prevent 100% of UV light from reaching the skin. Generally, the higher the SPF value, the more UV light the sunscreen prevents from absorbing into the skin. Therefore, even if the sunscreen prevents 99.5% of the UV light from penetrating the skin, some UV radiation is still making it through and naturally tanning will occur. This means if a consumer wears a gauge or device on the skin that is not essentially UV transparent, the consumer will have an untanned area of skin when the gauge or device is removed because the gauge or device is unable to transmit to the skin even the smallest amount of UV that is being transmitted by the sunscreen. It is a further object of the invention to provide an indicator that is essentially UV transparent.

Furthermore, gauges and devices attach to the skin or clothing using a separate adhesive layer. However, adhesive layers suffer from the drawback that they can fail causing the gauge or device to fall off and become useless. Therefore, it is a further object of this invention to provide an indicator that is applied directly to the skin, and that does not require a separate adhesive layer.

Another method for combating the problem of determining when a sunscreen has worn off is to put a temporary visual indicator in the sunscreen. The indicator renders the sunscreen colored when it is applied, but it becomes invisible shortly after application. The indicator can be reactivated temporarily to the visible form so that the user can verify the presence of the sunscreen. However, this method is inconvenient because the user must periodically reactivate the indicator by applying a disclosing solution, such as a spray to modify pH, to determine whether the sunscreen is still present. In addition, such methods rely on the consumer remembering to use the reactivation device. Therefore, it is a further object of this invention to provide a convenient method a consumer can use to automatically determine whether a sunscreen is still present on the skin.

Furthermore, many of the known indicators employ materials that change color irreversibly when exposed to UV radiation. These materials are not photochromic, according to Photochromism Molecules and Systems, ed. Heinz Dürr, Elsevier, 1990, but rather, undergo photochemistry, not photochromism. “Photochromic” means that the molecule changes color in the presence of radiation due to a conformational change of the molecule that is induced by radiation. This conformational change is a reversible structural change governed by mechanisms such as ring opening or closing reactions, electron transfer reactions, or radical dissociation reactions. For a molecule to be photochromic, it must be capable of reverting back to its original structure when exposure to the exciting radiation ceases. Furthermore, to be photochromic, the molecule must be capable of changing and reverting back to its original color, or nearly its original color, multiple times upon repeated exposures to radiation. Materials that change color irreversibly when exposed to UV radiation can be used to make disposable, one-time use dosimeter-type devices. However, they cannot be used to make reusable indicators (e.g., indicators that can be used upon multiple reapplications of sunscreen). Therefore, it is a further object of this invention to provide an indicator that can be used to determine whether a sunscreen is still providing effective and sufficient UV protection after one or more re-applications of the sunscreen over the course of one full day of outside activity.

### SUMMARY OF THE INVENTION

This invention relates to a sun protection kit comprising:

(A) a sunscreen, and

(B) an indicator comprising

(i) a photochromic molecule, and

(ii) a carrier.

The photochromic molecule turns or changes color when the indicator is exposed to ultraviolet radiation. The color change alerts the user when the sunscreen applied to the skin (over the indicator) is no longer acting as an effective sun protection product and needs to be reapplied. When the user reapplies the sunscreen, the photochromic molecule reverts to its original color, as does the indicator, because the sunscreen prevents the majority of the ultraviolet light from activating the photochromic molecule within the indicator. The kit preferably further comprises (C) instructions for use.

### DETAILED DESCRIPTION OF THE INVENTION

All percentages expressed herein are by weight unless otherwise indicated. All U.S. Patents cited herein are incorporated by reference in relevant part.

### Definitions and Usage of Terms

The following is a list of definitions for terms, as used herein:

“Aromatic group” means a monovalent group having a monocyclic ring structure or fused polycyclic ring structure. Monocyclic aromatic groups contain 5 to 12 carbon atoms, preferably 6 carbon atoms, in the ring. The most preferred monocyclic aromatic group is phenyl. Polycyclic aromatic groups contain 10 to 18 carbon atoms, preferably 10 to 12 carbon atoms in the ring system. Polycyclic aromatic groups include ring systems wherein only one ring in the system is aromatic. Polycyclic aromatic groups are exemplified by bicyclic groups of 10 to 12 carbon atoms and tricyclic groups of 16 to 18 carbon atoms. Preferred polycyclic aromatic groups include naphthyl, anthracenyl, and phenanthrenyl.

“Carbocyclic group” means a monovalent saturated or unsaturated hydrocarbon ring that is not aromatic. Carbocyclic groups can be monocyclic or polycyclic. Carbocyclic groups contain 5 to 18 carbon atoms, preferably 5 to 12 carbon atoms. Preferred monocyclic carbocyclic groups include cyclopentyl, cyclohexyl, and cyclohexenyl. Preferred polycyclic carbocyclic groups include bicyclic groups and tricyclic groups.

“Carrier” means a skin-like medium that keeps the photochromic molecule on the skin.

“Film-forming polymer” means a homopolymer, copolymer, or mixture thereof which forms a skin-adherent continuum from a composition when applied to skin. Film-forming polymer includes a monomer that will polymerize upon application to skin.

“Halogen atom” means F, Cl, Br, or I. Cl is preferred.

“Halogenated hydrocarbon group” means a substituted monovalent hydrocarbon group or a substituted carbocyclic group, wherein at least one substituent is a halogen atom.

“Heteroaromatic group” means an aromatic group containing carbon and 1 to 4 heteroatoms in the ring. Heteroaromatic groups are monocyclic or fused polycyclic rings. Monocyclic heteroaromatic groups contain 5 to 12 member atoms (i.e., carbon and heteroatoms), preferably 6 in the ring. Polycyclic heteroaromatic groups contain 10 to 18 member atoms, preferably 10 to 12 member atoms in the ring system. Polycyclic heteroaromatic groups include ring systems in which only one ring is aromatic. Polycyclic heteroaromatic groups are preferably bicyclic or tricyclic. Preferred bicyclic heteroaromatic rings include indolyl and quinolinyl.

“Heteroatom” means an atom other than carbon in the ring of a heterocyclic or heteroaromatic group or the chain of a heterogeneous group. Preferably, heteroatoms are selected from the group consisting of nitrogen, sulfur, and oxygen atoms. Groups containing more than one heteroatom may contain different heteroatoms.

“Heterocyclic group” means a saturated or unsaturated ring structure containing carbon and 1 to 4 heteroatoms in the ring. Heterocyclic groups are not aromatic. No two heteroatoms are adjacent in the ring. Heterocyclic groups can be monocyclic or polycyclic. Heterocyclic groups contain 5 to 18 member atoms, preferably 5 to 12 member atoms (i.e., including both carbon atoms and at least 1 heteroatom).

“Heterogeneous group” means a saturated or unsaturated chain containing 1 to 18 member atoms (i.e., including both carbon atoms and at least one heteroatom). “Lower heterogeneous” means a heterogeneous group having 1 to 6, preferably 1 to 3, member atoms. No two heteroatoms are adjacent. Preferably, the chain contains 1 to 12 member atoms. The chain may be straight or branched. Unsaturated heterogeneous groups have one or more double bonds, one or more triple bonds, or both.

“Monovalent hydrocarbon group” means a chain of 1 to 18, preferably 1 to 12 carbon atoms. “Lower monovalent hydrocarbon group” means a monovalent hydrocarbon group having 1 to 6, preferably 1 to 3 carbon atoms. Monovalent hydrocarbon groups may have a straight chain or branched chain structure. Unsaturated monovalent hydrocarbon groups have one or more double bonds, one or more triple bonds, or combinations thereof. Preferred monovalent hydrocarbon groups are lower monovalent hydrocarbon groups including alkyl groups such as methyl, ethyl, isopropyl, n-propyl, and t-butyl; and alkenyl groups such as vinyl and allyl.

“Skin-like” means that the indicator of this invention will behave similarly to skin in that sunscreen is applied to, absorbed by, and worn off the indicator in a similar manner as it does skin.

“Substituent” means a group which replaces hydrogen in the chemical structures described herein.

“Substituted aromatic group” means an aromatic group wherein 1 to 4 of the hydrogen atoms bonded to carbon atoms in the ring have been replaced with other substituents. Preferred substituents include: halogen atoms, cyano groups, acrylic groups, methacrylic groups, carboxy groups, alkoxy groups, amino groups, monovalent hydrocarbon groups, substituted monovalent hydrocarbon groups, heterogeneous groups, substituted heterogeneous groups, aromatic groups, substituted aromatic groups, heteroaromatic groups, substituted heteroaromatic groups, or any combination thereof.

“Substituted carbocyclic group” means a carbocyclic group wherein 1 to 4 hydrogen atoms bonded to carbon atoms in the ring have been replaced with other substituents. Preferred substituents include: halogen atoms, cyano groups, acrylic groups, methacrylic groups, carboxy groups, alkoxy groups, amino groups, monovalent hydrocarbon groups, substituted monovalent hydrocarbon groups, heterogeneous groups, substituted heterogeneous groups, aromatic groups, substituted aromatic groups, heteroaromatic groups, substituted heteroaromatic groups, or any combination thereof.

“Substituted heteroaromatic group” means a heteroaromatic group wherein 1 to 4 hydrogen atoms bonded to carbon atoms in the ring have been replaced with other substituents. The substituents include halogen atoms, cyano groups, acrylic groups, methacrylic groups, carboxy groups, alkoxy groups, amino groups, monovalent hydrocarbon groups, substituted monovalent hydrocarbon groups, heterogeneous groups, substituted heterogeneous groups, aromatic groups, substituted aromatic groups, heteroaromatic groups, substituted heteroaromatic groups, or any combination thereof.

“Substituted heterocyclic group” means a heterocyclic group wherein 1 to 4 hydrogen atoms bonded to carbon atoms in the ring have been replaced with other substituents. Preferred substituents include halogen atoms, cyano groups, acrylic groups, methacrylic groups, carboxy groups, alkoxy groups, amino groups, monovalent hydrocarbon groups, substituted monovalent hydrocarbon groups, heterogeneous groups, substituted heterogeneous groups, aromatic groups, substituted aromatic groups, heteroaromatic groups, substituted heteroaromatic groups, or any combination thereof.

“Substituted heterogeneous group” means a heterogeneous group, wherein 1 to 4 of the hydrogen atoms bonded to carbon atoms in the chain have been replaced with other substituents. Preferred substituents include halogen atoms, cyano groups, acrylic groups, methacrylic groups, carboxy groups, alkoxy groups, amino groups, monovalent hydrocarbon groups, substituted monovalent hydrocarbon groups, heterogeneous groups, substituted heterogeneous groups, aromatic groups, substituted aromatic groups, heteroaromatic groups, substituted heteroaromatic groups, or any combination thereof.

“Substituted monovalent hydrocarbon group” means a monovalent hydrocarbon group wherein 1 to 4 of the hydrogen atoms bonded to carbon atoms in the chain have been replaced with other substituents. Preferred substituents include halogen atoms, cyano groups, acrylic groups, methacrylic groups, carboxy groups, alkoxy groups, amino groups, monovalent hydrocarbon groups, substituted monovalent hydrocarbon groups, heterogeneous groups, substituted heterogeneous groups, aromatic groups, substituted aromatic groups, heteroaromatic groups, substituted heteroaromatic groups, or any combination thereof.

“Sunscreen” means any chemical product that absorbs or scatters UV radiation and prevents it from penetrating deep into the skin. Sunscreen includes sunblock products (e.g., products having an SPF greater than 15) and sunscreens and suntanning products (e.g., products having an SPF of 15 or less).

This invention relates to a sun protection kit comprising:

(A) a sunscreen, and

(B) an indicator comprising

(i) a photochromic molecule, and

(ii) a carrier,

wherein the carrier is essentially UV transparent, and wherein the indicator can be applied directly to skin without a separate adhesive layer. The kit preferably further comprises (C) instructions for use of the kit in the form of words, pictures, or both.

### Sunscreen

One of the advantages of this invention is that it can be used with sunscreens that are known in the art. Any sunscreen that prevents ultraviolet (“UV”) radiation from penetrating deep into the skin, and that does not destroy the efficacy of the indicator, is suitable for use in this invention.

Suitable sunscreens are commercially available. For example, COPPERTONE® products, such as COPPERTONE® SPF 8 and COPPERTONE® All Day Protection Sport SPF 48 are available from Schering-Plough HealthCare Products, Inc. of Memphis, Tenn. BANANA BOAT SPORT SUNBLOCK® Lotion SPF 15 is available from Sun Pharmaceuticals of Del Ray Beach, Fla. Suitable sunscreens are disclosed in U.S. Pat. No. 4,522,807.

Suitable sunscreens typically comprise active ingredients and inactive ingredients. Suitable active ingredients include those selected from the group consisting of benzophenone, ethylhexyl p-methoxycinnamate, 2-ethylhexyl salicylate, homosalate, octyl methoxycinnamate, octyl salicylate, oxybenzone, titanium dioxide, and combinations thereof.

Suitable inactive ingredients include those selected from the group consisting of acrylates such as C10 to C30 alkyl acrylate crosspolymer, alcohols, alkyl benzoate, aloe extract, aluminum hydroxide, aluminum starch octenylsuccinate, barium sulfate, benzyl alcohol, calcium panthotenate, camellia oleifera extract, carbomer, cetyl alcohol, cholecaciferol, diazolidinyl urea, dimethicone, disodium EDTA, FD&C dyes, fragrances, glycerin, iron hydroxide, isobutylparaben, isopropylparaben, isopropyl pareth carboxylate, jojoba oil, methylparaben, neopentyl glycol dioctanoate, octadecene/MA copolymer, panthenol, phenoxyethanol, phenoxysteareth-21, polyglyceryl-3 distearate, propylene glycol, propylparaben, retinyl palmitate, silica, sorbitan isostearate, sorbitol, stearic acid, triethanolamine, tocopherol acetate, trimethylpropate trioctanoate, water, and combinations thereof.

### Indicator

The indicator comprises a photochromic molecule and a carrier. The indicator may be rated for different SPFs and skin types, so consumers can choose to have an indicator that provides insight into different levels of protection.

The rating may be done, for example, by varying the amount of the photochromic molecule in the indicator. The indicator may comprise different levels of photochromic molecules in order to indicate when different SPF levels have been surpassed, such as in a tiered system which indicates that the sunscreen can no longer provide one level of SPF protection, but is still providing a lower level, e.g., the sunscreen is no longer providing SPF 30 protection but is providing SPF 15 protection, then that the sunscreen is no longer providing SPF15, but is SPF 10, etc.

The indicator may also be varied to provide signaling based on specific skin types. In the Fitzpatrick Skin Type Scale, skin type is based on the first 45-60 minutes of sun exposure after winter season without sunscreen protection. This scale is broken down into 6 types (Type I to Type VI). The Type I class is fair skin with freckles, and unexposed skin is white with a skin reaction that leads to easily-acquired and painful bums with little tan and ultimate peeling. Type VI is black or dark brown unexposed skin with bums resulting only from the most severe exposure (see Fitzpatrick, T. B., The validity and practicality of sun-reactive skin types I through VI, Arch. Dermatology 124 (1988) 869-871). The indicator may comprise different photochromic molecules and different levels of photochromic molecules to show when the sunscreen is providing adequate protection for one skin type but not others, e.g., the sunscreen is providing adequate protection for Type VI but not Type IV.

The indicator undergoes a color change when exposed to UV radiation. In a preferred embodiment of the invention, the indicator is colorless in the absence of UV radiation and becomes colored when exposed to UV radiation. Alternatively, the indicator may be partially colored in the absence of UV radiation, and may be changed (e.g., augmented or varied) by exposure to UV radiation. In an alternative embodiment of the invention, the indicator may be colored when not exposed to UV radiation, and become a different color when exposed to UV radiation. In another alternative embodiment of the invention, the indicator may be colored in the absence of UV radiation and colorless when exposed to UV radiation. The color (or lack thereof) upon exposure to UV radiation is determined by the selection of the photochromic molecule or molecules.

The indicator comprises a skin-like medium that keeps the photochromic molecule on the skin. “Skin-like” means that the indicator will behave in ways similar to the skin, so that the sunscreen is applied to, absorbed by, and worn off the indicator in a similar manner as it does on the skin. In terms of wearing off, the mechanism of removal or migration will be very similar on skin as on the indicator and with approximately the same rates.

However, the indicator is easily removed by wiping with common solvents, like rubbing alcohol or nail polish remover. The indicator is not easily removable with normal abrasion, soap and water, or both, to prevent removal upon swimming and casual washing and drying. Because there is no separate adhesive layer, there is essentially no feel to the indicator on the skin, so there is no distraction by the nagging of a sticker or secondary layer with an adhesive on the skin or by the visualization of a sticker or clear adhesive layer. The indicator is preferably basically invisible, both visually and tactilely. Therefore, any materials, such as TEGADERM® by 3M, which are visible and can be felt once applied, are not suitable for use in the carrier of the indicator of this invention.

In a preferred embodiment of the invention, the indicator is essentially transparent to radiation in both the UV and visible ranges, and the indicator is colorless in the absence of UV radiation. In this embodiment, this invention provides an added benefit in that it is not possible for the indicator to fall off, giving the user an added sense of security.

Furthermore, the carrier in the indicator must be essentially transparent in the ultraviolet region of the spectrum. Not only do sunscreens not block 100% of the UV light and therefore allow some tanning to occur, but as the sunscreen wears off, more UV light will begin to penetrate to the indicator. While some photons will be used to convert the photochromic molecule to its colored form, other photons will not and will need to pass through the indicator so that the skin under the indicator will tan. If the carrier is not essentially UV transparent, the patch of skin underneath the indicator (a.k.a., the area of skin to which the indicator is applied) will not tan as compared to the rest of the body which is naturally tanning in the sunlight, even with sun protection. The area of skin where the indicator will be a different color because less UV rays would have reached that area. Therefore, any materials, such as TEGADERM® by 3M, which are not essentially UV transparent, are not suitable for use in the indicator of this invention.

The indicator of this invention can be applied as either a solid film or as a fluid film. If it is applied as a solid film, it is pre-formed (e.g., a temporary tattoo) and is transferred to the skin from a backing layer that is only used to transfer the pre-formed film to the skin. When the indicator is a pre-formed film (e.g., the temporary tattoo), it is applied to the skin, for example, by pressing the film against the skin or wetting the film and thereafter pressing it against the skin.

The solid film indicator can have a variety of shapes, such as geometric shapes, odd shapes, letters and words, or figures (e.g., animals, cartoons, objects, and the like). Portions of the film may be colored with a design, which can be changed (e.g., augmented) by the color change of the photochromic molecule. The film may have areas with differing concentrations of the photochromic molecule such that the design will change as increasing areas become developed (i.e., change color as they are exposed to UV radiation). Alternatively, the photochromic molecule may be printed adjacent to colored reference zones which indicate the degree of exposure compared to the darkness of the areas in the film containing the photochromic molecule. Alternatively, the photochromic molecule may be in the shape of letters which spell a message when the photochromic molecule is exposed to UV radiation.

If the indicator is applied as a fluid film, it forms a thin film on the skin upon application. The forms of the indicator as a fluid film could encompass a balm stick, an ink pen or marker, a stamp-pad with stamper, a lotion, or the like, and combinations thereof.

Photochromic molecules

The indicator comprises a photochromic molecule and a carrier. Various classes of photochromic molecules are suitable for use in this invention. The classes include spirooxazines, spiropyrans, fulgides, fulgimides, bisimidazoles, and viologen derivatives, and combinations thereof. Spirooxazines, spiropyrans, and fulgides are known in the art, and are described in Photochromism Molecules and Systems, ed. Heinz Dürr, Elsevier, 1990. Examples of spirooxazines, their properties, and methods for their preparation are disclosed in pages 493-508. Examples of spiropyrans, their properties, and methods for their preparation are disclosed in pages 314-455. Examples of fulgides, their properties, and methods for their preparation are disclosed in pages 467-468.

Spirooxazines

“Spirooxazines” are molecules having a 3H oxazine ring in which the number 3 carbon atom of the ring is involved in a spiro linkage. The molecule contains condensed ring structural features such that the heterolytic cleavage of the carbon-oxygen bond results in a change of valence-bond structure and conformation of the molecule, as exemplified below.

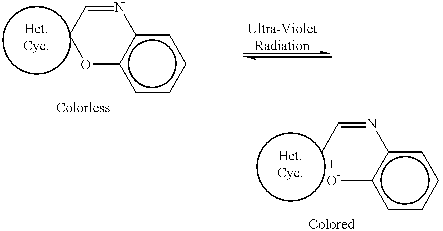

### Generalized Photochromic Reaction of Spirooxazines

Suitable spirooxazines for use in this invention have the general formula:

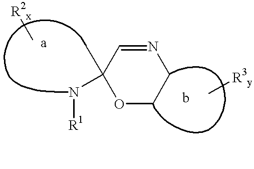

Group a is a spiro-linked group bonded to the carbon at the 3 position in the oxazine ring. Group a can be monocyclic or polycyclic. Group a is preferably bicyclic. Group a can be unsubstituted (i.e., when each R² is a hydrogen atom) or substituted (i.e., when at least 1 R² is not a hydrogen atom). Group a is preferably selected from the group consisting of aromatic groups, substituted aromatic groups, heteroaromatic groups, and substituted aromatic groups. In a preferred embodiment of the invention, group a is selected from the group consisting of heteroaromatic groups (e.g., indolyl, benzoxazolyl, and benzothiazolyl), and heterocyclic groups (e.g., piperidinyl groups).

Group b is a fused group bonded to the carbon atoms at the 5,6 position in the oxazine ring. Group b can be monocyclic or polycyclic. Group b is preferably bicyclic or tricyclic. Group b can be unsubstituted (i.e., when each R³ is a hydrogen atom) or substituted (i.e., when at least 1 R³is not a hydrogen atom). In a preferred embodiment of this invention, group b is selected from the group consisting of naphthyl, anthryl, and phenanthryl.

R¹ is a hydrogen atom or a lower monovalent hydrocarbon group. R¹ is preferably a hydrogen atom.

Each R² is independently a hydrogen atom, or a substituent group bonded to a carbon atom in the ring of group a. Substituent groups for R² are exemplified by monovalent hydrocarbon groups, substituted monovalent hydrocarbon groups, carbocyclic groups, substituted carbocyclic groups, heteroaromatic groups, substituted heteroaromatic groups, heterogeneous groups, substituted heterogeneous groups, a hydroxyl group, and an amino group. Preferred heterogeneous groups include alkoxy groups, such as methoxy and ethoxy groups, and cyano groups.

Each R³ is independently a hydrogen atom, a monovalent hydrocarbon group, an amino group, a substituted monovalent hydrocarbon group (e.g., alkoxy groups, such as methoxy and ethoxy groups, and cyano groups), a heterogeneous group, or a substituted heterogeneous group.

The subscript x represents the total number of hydrogen atoms and substituent groups bonded to carbon atoms in the ring of group a.

The subscript y represents the total number of hydrogen atoms and substituent groups bonded to carbon atoms in the ring of group b.

Suitable spirooxazines for use in this invention are typically selected from the group consisting of:

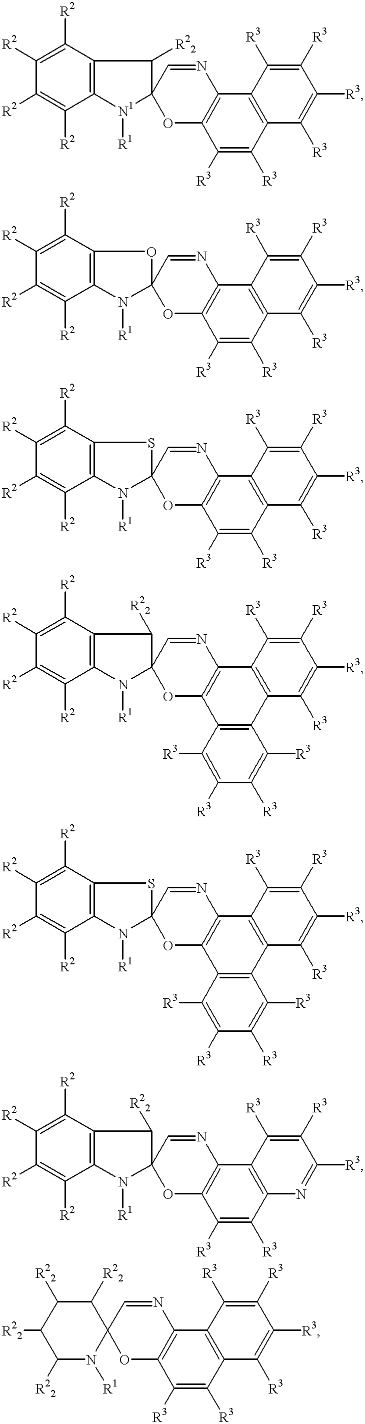

R¹, R² and R³ are as described above.

Spiro(indoline-2,3′-(3H)naphth(2,1-b)(1,4)oxazines) have the formula:

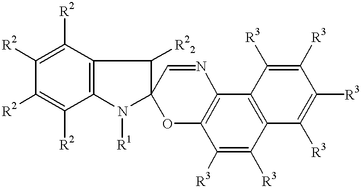

wherein R¹, R², and R³ are as described above. Examples of spiro(indoline-2,3′-(3H)naphth(2,1-b)(1,4)oxazines) include 1,3,3-trimethyl spiro(indoline-2,3′-(3H)naphth(2,1-b)(1,4)oxazine), and 3-ethyl-9′methoxy-1,3-dimethylspiro(indoline-2,3′-(3H)naphth(2,1-b)(1,4)oxazine).

Spiro(indoline-2,3′-(3H)pyrido(3,2-f)(1,4)benzoxazines) have the formula:

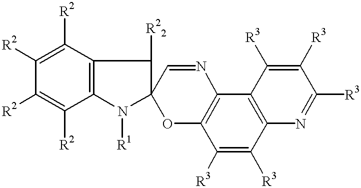

wherein R¹, R², and R³ are as described above. Suitable spiro(indoline-2,3′-(3H)pyrido(3,2-f)(1,4)benzoxazines) include 1,3,3-trimethylspiro(indoline-2,3′-(3H)pyrido(3,2-f)-(1,4)benzoxazine) and 1,3-dihydrospiro(indoline-2,3′-(3H)pyrido(3,2-f)-(1,4)benzoxazine).

Spiropyrans

“Spiropyrans” are molecules having two heterocyclic parts linked together by a common tetrahedral sp³ carbon atom. The two heterocyclic parts are in different orthogonal planes. A benzopyran structure in at least one heterocyclic part is common to all spiropyrans. Spiropyrans undergo a heterolytic cleavage of the carbon oxygen bond similar to that of spirooxazines. Photochromic interconversion of a spiropyran is exemplified below.

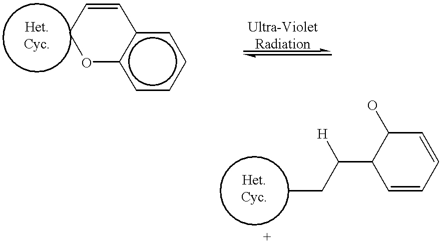

Spiropyrans suitable for use in this invention have the following general formula.

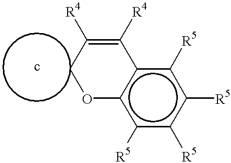

Group c is a spiro-linked group selected from the group consisting of heterocyclic, substituted heterocyclic groups, heteroaromatic groups, and substituted heteroaromatic groups. Group c is preferably selected from the group consisting of azaheterocyclic groups and nonazaheterocyclic groups. Azaheterocyclic groups include indoline; benzothiazoline; benzoxazoline; benzoselenazoline; 1,3-thiazolidine; 1,3-oxazolidine; pyrrolidine; 1,3-thiazine; 1,4-thiazine; 1,3-oxazine; piperidine; phenanthridine; acridine; and quinoline. Nonazaheterocyclic groups include benzodithiole, benzoxathiole, benzopyran, naphthopyran, xanthene, and dithiole.

Each R⁴ is independently selected from the group consisting of a hydrogen atom, monovalent hydrocarbon groups (e.g., alkyl groups), substituted monovalent hydrocarbon groups, heterogeneous groups (e.g., alkoxy, alkoyl, halogenated hydrocarbon groups, and carboxy groups), substituted heterogeneous groups, aromatic groups (e.g., phenyl, and naphthyl), substituted aromatic groups, heteroaromatic groups, substituted heteroaromatic groups, cyclic groups, substituted cyclic groups (e.g., cycloalkyl groups), heterocyclic groups, substituted heterocyclic groups, nitro groups, hydroxy groups, and amino groups.

Each R⁵ is independently selected from the group consisting of R⁴ and with the proviso that two instances of R⁵ may be bonded together to form a fused group selected from the group consisting of aromatic groups (e.g., phenyl, and naphthyl), substituted aromatic groups, heteroaromatic groups, substituted heteroaromatic groups, cyclic groups, substituted cyclic groups (e.g., cycloalkyl groups), heterocyclic groups, substituted heterocyclic groups, nitro groups, hydroxy groups, and amino groups.

In a preferred embodiment of the invention, the spiropyrans are naphthopyrans. Naphthopyrans have the following formula:

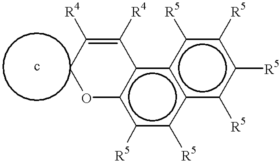

wherein group c, R⁴, and R⁵ are as described above.

Examples of suitable spiropyrans to use in this invention include 1′,3′,3-trimethylspiro(2H-1)benzopyran-2,2′-indoline, 6-nitro-1′,3′,3′-trimethylbenzoindolinospiropyran, 3-ethyl-8-methoxy-6-nitro(2H-1)benzopyran-2-spiro-2′(3′-methylthiazolidine), 8-bromo-6-nitro-indolinospirobenzopyran, and 8-nitro-indolinospirobenzopyran.

Fulgides

“Fulgides” are dimethylene succinic anhydride derivatives. Fulgides convert from a colorless to a colored form upon exposure to UV radiation. Unlike the spirooxazines and spiropyrans, the colored form is a closed ring system, and the colorless form results from a ring opening reaction.

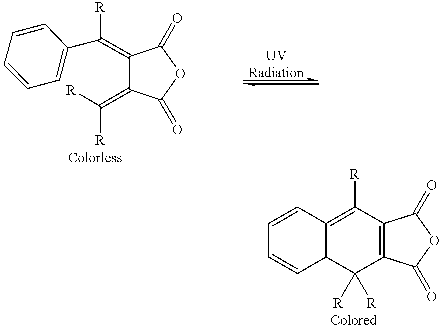

### Generalized Photochromic Reaction of Mono-aryl Fulgides

Fulgides suitable for use in this invention include those having the general formula:

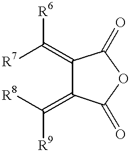

wherein R⁶, R⁸ and R⁹ are each independently selected from the group consisting of a hydrogen atom, a monovalent hydrocarbon group (e.g., an alkyl group such as methyl or ethyl), a substituted monovalent hydrocarbon group, a heterogeneous group (e.g., a halogenated hydrocarbon group, alkoxy groups), and a substituted heterogeneous group.

R⁷ is selected from the group consisting of an aromatic group, a substituted aromatic group (e.g., phenyl, phenoxy, or alkoxyaryl), a heteroaromatic group, a substituted heteroaromatic group, a carbocyclic group, a substituted carbocyclic group, a heterocyclic group (e.g., thienyl and furanyl), and a substituted heterocyclic group (methylthienyl and methylfuranyl).

Examples of suitable fulgides for use in this invention include alkoxyaryl fulgides such as methoxyaryl fulgides and heterocyclic fulgides such as furyl fulgides and adamantylidene fulgides.

Suitable alkoxyaryl fulgides are exemplified by the following:

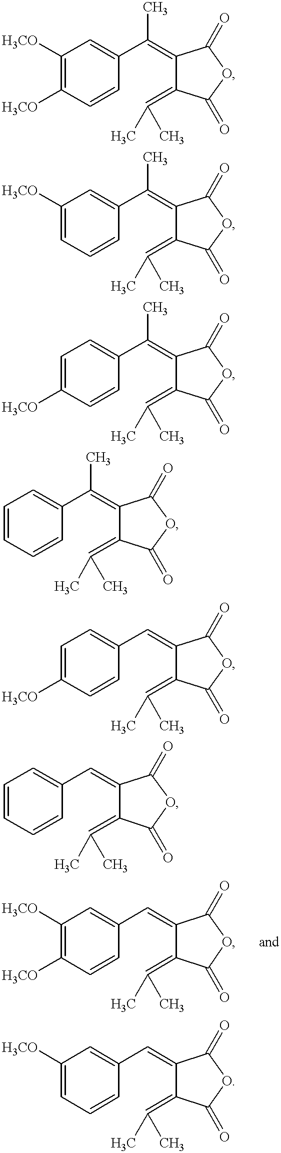

Suitable heterocyclic fulgides include those selected from the group consisting of:

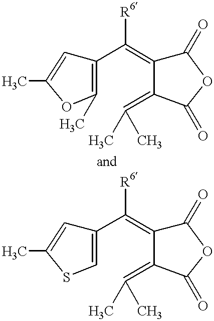

wherein R⁶ is an alkyl group such as methyl, ethyl, n-propyl, or isopropyl.

Fulgimides

Fulgimides are similar to the fulgides described above. Fulgimides have the general formula:

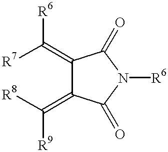

wherein R⁶, R⁷, R⁸ and R⁹ are as described above. Fulgimides are exemplified by:

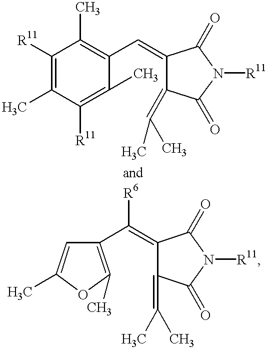

wherein R⁶ is as described above and each R¹¹ is independently selected from the group consisting of a hydrogen atom, a monovalent hydrocarbon group (e.g., an alkyl group such as methyl or ethyl), a substituted monovalent hydrocarbon group, a heterogeneous group (e.g., a halogenated hydrocarbon group, alkoxy groups), and a substituted heterogeneous group.

Bisimidazoles

The UV-induced photochromic mechanism of bisimidazoles is a radical dissociation. Suitable bisimidazoles have the following general formula:

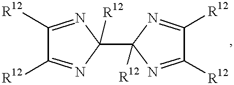

wherein each R¹² is independently selected from the group consisting of an aromatic group, a substituted aromatic group (e.g., phenyl, phenoxy, or alkoxyaryl), a heteroaromatic group, and a substituted heteroaromatic group. Preferably, R¹² is selected from the group consisting of a phenyl group, a methylphenyl group, a methoxyphenyl group, and a halogenated phenyl group.

Viologen Derivatives

Viologen derivatives are also suitable to use as the photochromic molecule of this invention. The photochromic mechanism of viologen takes place via an electron transfer reaction between the halogen and the nitrogen atoms. Viologen derivatives have the general formula:

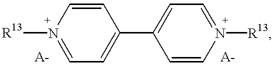

wherein each R¹³ is independently selected from the group consisting of a monovalent hydrocarbon group (e.g., an alkyl group such as methyl or ethyl), a substituted monovalent hydrocarbon group, a heterogeneous group (e.g., a halogenated hydrocarbon group, alkoxy groups), a substituted heterogeneous group, an aromatic group, a substituted aromatic group (e.g., phenyl, phenoxy, or alkoxyaryl), a heteroaromatic group, a substituted heteroaromatic group, a carbocyclic group, a substituted carbocyclic group, a heterocyclic group (e.g., thienyl and furanyl), and a substituted heterocyclic group (methylthienyl and methylfuranyl); and each A represents a halogen atom. Suitable photochromic molecules are known in the art and are commercially available. For example, PPG Industries, Inc. of Monroeville, Pa. sells a line of photochromic dyes that can be activated (i.e., change color) by exposure to ultraviolet radiation having a wavelength of 340 to 360 nanometers. These photochromic dyes include PHOTOSOL® 0265, 33672, 5-3 (a spirooxazine), 5-83, 7-106, and 7-49.

Photochromic molecules in a variety of colors are suitable for this invention. However, of the PHOTOSOLS®, preferred photochromic molecules are blue or purple when exposed to UV radiation and colorless when not exposed. Generally, the blue and purple PHOTOSOLS® revert back to their colorless form more quickly than red and pink. Furthermore, PHOTOSOLS® that turn pink and red when exposed to UV radiation absorb some UV radiation having wavelengths of 360 to 420 nanometers. Many sunscreens do not block UV radiation having wavelengths greater than 365 nanometers. Therefore, photochromic molecules exhibiting a color change when exposed to UV radiation having wavelengths of 365 nanometers or less are preferred. Examples of commercially available photochromic molecules and their colors are in Table 1.

TABLE 1
Commercially Available Photochromic Molecules

| Photochromic Molecule | Color |
|---|---|
| PHOTOSOL ® 0265 | blue |
| PHOTOSOL ® 33672 | blue |
| PHOTOSOL ® 5-3 | yellow |
| PHOTOSOL ® 5-83 | orange |
| PHOTOSOL ® 7-106 | purple |
| PHOTOSOL ® 7-49 | red |

In an alternative embodiment of the invention, an irreversible molecule (i.e., one that undergoes an essentially irreversible color change on exposure to UV radiation) can be used to make a dosimeter-type indicator. In the dosimeter-type indicator, the irreversible molecule is substituted for some or all of the photochromic molecule, which is used in the preferred embodiments of the invention. Suitable irreversible molecules include 18-molybdodiphosphoric acid, oxazolidine-dione compounds, xanthenone compounds, tetrazolium salts, photosensitive onium salts and dyes which change color when protonated, and 2(2′,4′dinitrobenzy)pyridine, and combinations thereof. Suitable irreversible molecules are diclosed in U.S. Pat. Nos. 5,589,398 and 5,117,116. The dosimeter-type indicator can be used to measure cumulative UV exposure.

Carriers

The indicator further comprises a carrier. “Carrier” means a skin-like medium that keeps the photochromic molecule on the skin. Suitable carriers are disclosed in PCT Publication Nos. WO 99/56705 and WO 99/55290. The carrier can be a composition comprising one or more components. Carriers are self-adhering to skin. Carriers do not have a separate adhesive layer.

The carrier composition comprises a (i) film-forming polymer, (ii) a liquid diluent, and (iii) one or more suitable optional components described herein. As used herein, the term “film-forming polymer” means a homopolymer, copolymer, or mixture thereof which forms an adherent continuum from a composition when applied to skin. See, e.g., Polymer Colloids, ed. Robert M. Fitch, Plenum Press, 1971. As used herein, the term “copolymer” includes linear, block, branched, graft, comb, and star copolymers.

(i) Film-Forming Polymer

Although the term “film-forming polymer” is used herein to describe a polymer in a composition, in some circumstances, polymerization may not actually take place until application of the composition (to the skin, for example) is performed. Accordingly, as used herein, the term “film-forming polymer” is meant to encompass monomers which have not yet polymerized but will upon application to the skin.

The film-forming polymers herein are preferably self-curing polymers. That is, the preferred polymers do not require chemical reaction or introduction of energy (e.g., exposure to UV rays) to form the adherent continuum.

The film-forming polymers herein can be selected from nonionic, ionic (anionic or cationic), and amphoteric (including zwitterionic) polymers. When the film-forming polymer is water-borne, the polymer is preferably anionic.

Film-forming polymers can be natural polymers and derivatives of natural polymers or synthetic polymers and copolymers. Examples of natural polymers are xanthan gum (bacteriological gum), seaweed (alginates), tree gums (guar), chitins, starches, pectins, proteins, cellulosic polymers, and the like. Examples of synthetic polymers are polyurethanes, polysiloxanes, polyacryls, and the like.

The film-forming polymers herein are preferably, but are not limited to, solvent-borne, water-borne, or water-soluble polymers. As used herein, the term “water-borne”, with reference to a film-forming polymer, means that the polymer was prepared in a mixture comprising water and is preferably added to the composition which it comprises as a mixture (preferably a dispersion) in water. As used herein, the term “water-soluble”, with reference to a film-forming polymer, means that the polymer was prepared in water, and mixed so as to dissolve the polymer in water. As used herein, the term “solvent-borne”, with reference to a film-forming polymer, means that the polymer was prepared under substantially anhydrous conditions and is preferably added to the composition which it comprises as a substantially anhydrous mixture (preferably a solution).

Preferred film-forming polymers of the present invention are selected from the group consisting of polyurethanes, polyacryls, polymethacryls, cellulosic polymers, styrene-acryl copolymers, polystyrene-polyacryl mixtures, polysiloxanes, polyesters, urethane-acryl copolymers, siloxane-urethane copolymers, polyurethane-polymethacryl mixtures, silicone-acryl copolymers, vinyl acetate polymers, fluoropolymers, and mixtures thereof. The term “polyacryl” includes polyacrylates, polyacrylics, and polyacrylamides. The term “polymethacryl” includes polymethacrylates, polymethacrylics, and polymethacrylamides. The term “cellulosic polymers” includes all cellulose polymers, including esters thereof.

Examples of preferred polyacryls, polymethacryls, and styrene-acryl copolymers include GELVA® 2497 (commercially available from Monsanto Co., St. Louis, Mo.), DURAPLUS® 2 (commercially available from Rohm & Haas Co., Philadelphia, PA), JONCRYL® 95, SCX-1537, SCX-1965, SCX-1959, JONCRYL® 530, JONCRYL®537 (all commercially available from S.C. Johnson Polymer, Sturtevant, Wis.), GLASCOL® LS20, GLASCOL® C37, GLASCOL® LS26, GLASCOL(® LS24, GLASCOL® LE45, SURCOL® 441 (all commercially available from Allied Colloids, Suffolk, Va.), CARBOSET® CR760, CARBOSET® CR761, CARBOSET® CR763, CARBOSET® 765, CARBOSET® 19X2, CARBOSET® XL28, HYCAR® 26084, HYCAR® 26091, CARBOBOND® 26373 (all commercially available from B. F. Goodrich, Cleveland, Ohio), NEOCRYL® A-601, NEOCRYL® A-612, NEOCRYL® A-6044, NEOCRYL® A-622, NEOCRYL® A-623, NEOCRYL® A-634, AND NEOCRYL® A-640 (all commercially available from Zeneca Resins, Wilmington, Mass.).

Examples of preferred urethane-acryl copolymers include SANCURE® AU-4000, SANCURE® AU-4010 (both commercially available from B. F. Goodrich), WITCOBOND® A-100, WITCOBOND® W-610 (both commercially available from Witco Performance Chemicals, Houston, Tex.), NEOPAC® R-9000, NEOPAC® R-9030, NEOPAC® R-9699 (Zeneca Resins).

Preferred polyurethanes are selected from aromatic polyether polyurethanes, aliphatic polyether polyurethanes, aromatic polyester polyurethanes, aliphatic polyester polyurethanes, aromatic polycaprolactam polyurethanes, and aliphatic polycaprolactam polyurethanes. The more preferred polyurethanes are selected from aromatic polyether polyurethanes, aliphatic polyether polyurethanes, aromatic polyester polyurethanes, and aliphatic polyester polyurethanes. Examples of preferred polyurethanes include SANCURE 2710®, AVALURE UR 445® (which are equivalent copolymers of polypropylene glycol, isophorone diisocyanate, and 2,2-dimethylolpropionic acid, having the International Nomenclature Cosmetic Ingredient name “PPG-17/PPG-34/IPDI/DMPA Copolymer”), and combinations thereof, SANCURE 878®, SANCURE 815®, SANCURE 1301®, SANCURE 2715®, SANCURE 1828®, SANCURE 2026®, SANCURE 1818®, SANCURE 853®, SANCURE 830®, SANCURE 825®, SANCURE 776®, SANCURE 850®, SANCURE 12140®, SANCURE 12619®, SANCURE 835®, SANCURE 843®, SANCURE 898®, SANCURE 899®, SANCURE 1511, SANCURE 1514®, SANCURE 1517®, SANCURE 1591®, SANCURE 2255®, SANCURE 2260®, SANCURE 2310®, SANCURE 2725®, and SANCURE 12471® (all commercially available from B. F. Goodrich), BAYHYDROL® LS-2033, BAYHYDROL® DLN, BAYHYDROL® 123, BAYHYDROL® PU402A, BAYHYDROL® 110 (all commercially available from Bayer Corp., McMurray, Pa.), WITCOBOND® W-320, WITCOBOND® W-242, WITCOBOND® W-160, WITCOBOND® W-612, and WITCOBOND® W-506 (all commercially available from Witco Performance Chemicals), NEOREZ® R-940, NEOREZ® R-960, NEOREZ® R-962, NEOREZ® R-966, NEOREZ® R-967, NEOREZ® R-972, NEOREZ® R-9409, NEOREZ® R-9637, NEOREZ® R-9649, and NEOREZ® R-9679 (all commercially available from Zeneca Resins).

Preferred solvent-borne polyurethanes include SANRES EX499®, (hexylene glycol/neopentyl glycol/isophorone diisocyanate copolymer), SANRES 12711®, SANRES 6010®, and SANRES 6012® (all commercially available from B. F. Goodrich). The most preferred solvent-borne polyurethane is SANRES EX499®.

Examples of water-borne polyurethanes include SANCURE® 2060 and SANCURE® 815 (both commercially available from B. F. Goodrich).

The most preferred water-borne polyester polyurethanes are aliphatic polyether polyurethanes. Examples of preferred aliphatic polyether polyurethanes include SANCURE® 2710, and/or AVALURE UR 445®, SANCURE® 878, NEOREZ® R-966, NEOREZ® R-967, and WlTCOBOND® W-320.

Preferred cellulosic polymers include, for example, nitrocellulose, nitrocellulose esters, such as cellulose acetate, cellulose acetate butyrate, and cellulose acetate propionate and mixtures thereof. The most preferred cellulosic polymer is nitrocellulose. Exemplary nitrocellulose polymers are nitrocellulose RS types (nitrogen content of 11.5% to 12.2%), commercially available from Hercules, such as nitrocellulose RS ½ second, nitrocellulose RS ¼ second, nitrocellulose RS 1/8 second, and nitrocellulose RS 1/16 second, and the like. Where the composition comprises a cellulosic polymer, the composition preferably further comprises a plasticizer.

The film-forming polymer can also be a silicone gum. Suitable silicone gums are exemplified by those disclosed in PCT Patent Publication Nos. WO 97/17058 and WO 97/17059. Suitable silicone gums have viscosities in the range of 100,000 to 2,500,000 centistokes at 25° C. Suitable silicone gums are exemplified by dimethicone gums and phenyl methyl silicone gums. Dimethicone gums are known in the art and commercially available as SE® 30, SE® 63, and SE® 90 from General Electric Company of Schenectady, N.Y. Phenyl methyl silicone gum is available as 88778, also from General Electric.

(ii) Liquid Diluent

The compositions of the present invention further comprise a liquid diluent. The liquid diluent can comprise a solvent, other diluents, or combinations thereof. The solvent can be water or an organic solvent. Preferred organic solvents include those which are volatile. Preferred volatile organic solvents, at atmospheric pressure, have a boiling point of from about 50° C. to about 140° C., more preferably from about 56° C. to about 125° C., and most preferably from about 56° C. to about 98° C. Wherein the film-forming polymer utilized is water-borne, the organic solvent is preferably water-miscible.

Non-limiting examples of suitable organic solvents are n-amyl acetate, n-propyl acetate, acetone, heptane, isobutyl acetate, isopropyl acetate, toluene, methyl acetate, ethyl acetate, alcohols (e.g., ethanol, isopropanol, isobutanol, n-amyl alcohol, and n-butyl alcohol), hydrocarbons (e.g., hexane and isododecane), ketones such as methyl ethyl ketone, ethyl oleate, glycerin, glycols (e.g., polypropylene glycol and polyethylene glycol), buffer solutions (e.g., phosphate, potassium acetate, boric carbonic, phosphoric, succinic, malic, tartaric, citric, acetic, benzoic, lactic, glyceric, gluconic, glutaric and glutamic), esters, methylene chloride, castor oil, ethers, dimethylsulfoxide, dimethyl formamide, tetrahydrofuran, and combinations thereof.

The preferred organic solvents are selected from alcohols, esters, ketones, aromatic hydrocarbons, aliphatic hydrocarbons, ethers, and mixtures thereof. Alcohols, esters, and hydrocarbons are more preferred. Preferred alcohols are monohydric. The most preferred monohydric alcohols are ethanol, isopropanol, and n-propanol. The most preferred esters are ethyl acetate, isopropyl acetate, and butyl acetate. Preferred hydrocarbons are hexane and isododecane.

The liquid diluent typically comprises about 10% to about 90%, preferably about 15% to about 80%, more preferably about 20% to about 70%, of the carrier composition. The preferred liquid diluents are toxicologically acceptable and safe for use in contact with skin.

(iii) Optional Components

The compositions of the present invention may, independently, comprise additional optional components to enhance their performance as a indicator. For example, coalescents, coloring agents such as pigments and dyes, plasticizers, preservatives, resins, gelling agents, stabilizers, chelating agents, dispersing agents, fillers, thickeners, wax additives, prophylactic agents, wetting agents, combinations thereof, and the like can be included in the compositions herein. Such optional components may be dispersed, solubilized, or otherwise mixed in the carrier of the compositions. These components may be added to the compositions herein provided they do not substantially hinder the performance of the films formed from the compositions and of the indicators. Non-limiting examples of some of the above optional components are given below.

Coalescents

Coalescents may optionally be added to the compositions to enhance film-formation, most preferably wherein the film-forming polymer is water-borne. Coalescents are coalescing aids that are known in the art and are typically glycol ethers or glycol ether esters such as C₁₋₁₀ straight or branched chain alkyl glycol alkyl ethers, C₁₋₁₀ straight or branched chain alkyl ether acetates, di-C₁₋₁₀ alkyl ether acetates, C₁₋₁₀ alkyl glycol phenyl ethers, tripropylene glycol ethers, terpenes, camphor, methyl cellusolve, butyl cellusolve, hexyl cellusolve, methyl carbitol, and butyl carbitol. Preferred coalescing aids include, for example, ethylene glycol ethers (e.g., DOWANOL EB®, commercially available from Dow Chemical Co., Midland, Mich.), diethylene glycol ethers, triethylene glycol ethers, propylene glycol ethers (e.g., DOWANOL PNP®, Dow Chemical Co.), dipropylene glycol ethers (e.g., DOWANOL DPNP®, Dow Chemical Co.), tripropylene glycol ethers, terpenes, camphor, methyl cellusolve, butyl cellusolve, hexyl cellusolve, methyl carbitol, and butyl carbitol.

Preferably, these compositions comprise from 0% to about 10%, by weight of the composition, of a coalescent.

Coloring Agents

Pigments and other suitable coloring agents, such as dyes, may be incorporated into the compositions, depending on the desired properties of the indicator. Suitable pigments are inorganic or organic pigments known as, for example, the FD&C and D&C colors, lakes, and iron oxides. Such pigments are disclosed in the C.T.F.A Cosmetic Ingredient Handbook, First Edition, 1988. Organic pigments include, for example, D and C Red, Nos. 10, 11, 12, and 13, D and C Red No. 7, D and C Red Nos., 5 and 6, D and C Red Nos. 30 and 34, lacquers such as D and C Yellow No. 5, and D and C Red No. 2, and guanine. Inorganic pigments include, for example, titanium dioxide, bismuth oxychloride, brown iron oxide, and the red iron oxides.

Preferably, the present compositions comprise from 0% to about 5% and most preferably from 0% to about 2%, by weight of the composition of a coloring agent.

Plasticizers

Without intending to be limited by theory, plasticizers cause a composition to become more easily deformed. One or more plasticizers may optionally be added to the present compositions. Suitable plasticizers include those disclosed in PCT Patent Publication No. WO 97/00664. Suitable plasticizers include phthalates, tartrates, phosphates, tetraethylene glycol di-2-ethylhexoate, nonionic surfactant polymers, camphor, castor oil, sucrose acetate isobutyrate, alkyl toluenesulfonamides, e.g., ethyl toluenesulfonamide (e.g., Uniplex PX-45, commercially available from Unitex Chemical Corp., Greenboro, N.C.), and polyester acid derivatives (e.g., Uniplex 670P, also commercially available from Unitex Chemical Corp.), particularly polyester di- and tri-acids. Preferred plasticizers include diethyl phthalate, dibutyl phthalate, dioctyl phthalate, diethyl tartrate, dibutyl tartrate, diethyl phosphate, dibutyl phosphate, polyester sebacates, such as PARAPLEX G-25®, polyester adipates, such as PARAPLEX G-50®, and tetraethylene glycol di-2-ethylhexoate, available as TEGMER® (all commercially available from C. P. Hall, Bedford Park, Ill.). The most preferred plasticizers include dibutyl phthalate, PARAPLEX G-25®, PARAPLEX G-50®, camphor, Uniplex PX-45, and TEGMER®.

A composition preferably comprises from 0% to about 15%, more preferably from 0% to about 10% and most preferably from 0% to about 5%, by weight of the composition, of a plasticizer.

Preservatives

One or more preservatives may optionally be added to the present compositions to prevent, inhibit, or retard microbial growth in the composition. Preferred preservatives include methyl paraben, ethyl paraben, propyl paraben, phenols, alkyl esters of parahydroxybenzoic acid, benzoic acid and the salts thereof, benzoates (preferably sodium benzoate), boric acid and the salts thereof, sorbic acid and the salts thereof, sorbates, (preferably potassium sorbate), chorbutanol, thimerosal, phenylmercuric acetate and nitrate, nitromersol, benzalkonium chloride, cetylpyridinium chloride, benzyl alcohol sodium dehydroacetate, 1-(3-chloroallyl)-3,5,7-triaza-1-azoniaadamantane chloride (which may be obtained commercially as QUATERNIUM-15® from Dow Chemical Co., Midland, Mich.), a mixture of 95% 1,3-dimethylol-5,5-dimethyl hydantoin and 5% 3-iodo-2-propynyl butyl carbamate (which is commercially available as GLYDANT PLUS® from Lonza, Inc., Fair Lawn, N.J.), 1,3-dimethylol-5,5-dimethyl hydantoin (commercially available as GLYDANT® from Lonza, Inc.), diazolidinyl urea (commercially available as GERMALL II® from Sutton Laboratories, Chatham, N.J.), imidazolidinyl urea (commercially available as GERMALL 115® from Sutton Laboratories), and phenoxyethanol, and KATHON® (commercially available from Rohm and Haas Co., Philadelphia, Pa.). The most preferred preservatives include methyl paraben, ethyl paraben, propyl paraben, benzyl alcohol, benzoic acid, the salts of benzoic acid, benzoates (preferably sodium benzoate), cetylpyridinium chloride, sorbates (preferably potassium sorbate), and sodium dehydroacetate.

A composition preferably comprises from 0% to about 10%, more preferably from 0% to about 5%, and most preferably from 0% to about 1%, by weight of the composition, of a preservative.

Resins

Resins including, for example, epoxies and polyacrylics, may optionally be added. Examples of suitable resins include, but are not limited to, POLYTEX E75® (commercially available from Estron Chemical, Inc., Calvert City, Ky.) and ACRYLOID B66(® (commercially available from Rohm and Haas, Philadelphia, Pa.).

Other suitable resins include organosiloxane resins which comprise units of the formulae: R¹⁰₃SiO_{1/2} (“M” units”), R¹⁰₂SiO_{2/2} (“D” units), R¹⁰SiO_{3/2} (“T” units), and R¹⁰SiO_{4/2} (“Q” units). The amounts of each unit satisfy the relationship R¹⁰ₙSiO_{(4-n)/2}, wherein n is a number with a value of 1.0 to 1.5, preferably 1.2. Each R¹⁰ is independently selected from the group consisting of monovalent hydrocarbon groups (e.g. alkyl groups such as methyl and ethyl groups), substituted monovalent hydrocarbon groups, heterogeneous groups, substituted heterogeneous groups, carbocyclic groups, substituted carbocyclic groups, heterocyclic groups, substituted heterocyclic groups, aromatic groups, substituted aromatic groups, heteroaromatic groups, and substituted heteroaromatic groups. R¹⁰ is preferably a monovalent hydrocarbon group, more preferably an alkyl group, most preferably a methyl group.

The organosiloxane resin may contain a small amount (i.e., up to about 5%) of silanol or alkoxy groups for R¹⁰. The organosiloxane resin is preferably solid at 25° C. and has a molecular weight of 1,000 to 10,000 g/mole. The resin is preferably soluble in organic solvents such as toluene, isoparaffins, and cyclosiloxanes. Preferred resins are MQ resins (i.e., resins comprising primarily M and Q units). The ratio of M:Q units is preferably about 0.6:1 to 0.9:1, preferably about 0.7:1.

Suitable organosiloxane resins are known in the art and are commercially available. Suitable organosiloxane resins include WACKER® 803 and 804 from Wacker Silicones Corporation of Adrian, Mich. and GE® SR 100MQ and GE® 1170-002 from General Electric Company of Schenectady, N.Y.

The composition preferably comprises from 0% to about 40%, more preferably from about 0.5% to about 30%, by weight of the composition, of the resin.

Gelling Agents

Suitable gelling agents are known in the art and are commercially available. Suitable gelling agents are available from Rheox, Inc., of Hightstown, N.J.; and these gelling agents include BENTONE® products. For example, BENTONE® EW theological additive is a processed hectorite clay gellant. BENTONE® MA rheological additive is a purified hectorite which thickens, forms gels, and suspends solids in aqueous systems. BENTONE® LT theological additive is a mineral (hectorite) product.

The amount of gelling agent in the composition is typically 0% to about 20% and preferably from about 1.0% to about 15%.

Stabilizers

One or more stabilizers may be added to the compositions herein, e.g., to prevent pigment from settling, to minimize or prevent photo- or other degradation of the photochromic molecule, or to achieve desired application properties. Preferably, stabilizers are added to compositions comprising a solvent-borne film-forming polymer. Preferred stabilizers include organically modified clays, e.g., organically modified bentonites and hectorites such as stearalkonium bentonite and stearalkonium hectorite (commercially available from Rheox, Inc., Uighstown, N.J.). Other suitable stabilizers include butylated hydroxyanisole, butylated hydroxytoluene, and vitamin E.

Wherein a stabilizer is added, the composition preferably comprises from about 0.25% to about 3%, still more preferably from about 0.25% to about 2.5%, and most preferably from about 1% to about 2% of the stabilizer, by weight of the composition.

Other optional ingredients may be added as long as they do not interfere with the function of the photochromic molecule. One skilled in the art would be able to select appropriate optional ingredients without undue experimentation.

Physical Characteristics of the Compositions

The compositions of this invention, when applied to the skin, will provide films that exhibit good adhesion, high flexibility, and fast drying rates. These characteristics can be defined by physical parameters such as surface energy, Young's Modulus, glass transition temperature, and drying rate, to name a few. The following parameters are cited as non-limiting examples of characteristics that can be used when choosing the film-forming polymer and diluent. The physical parameters described herein do not limit the scope of film-forming polymers suitable to use in this invention. Other properties can be used as well to give to the desired characteristics.

a. Surface energy: Good adhesion can be a result of matching the surface energy (γₛ) of the skin with that of the composition. Such adhesion is believed to be due to physical forces, rather than chemical bonding to the skin. As is known in the art, these physical forces include non-covalent interactions such as polar, non-polar, hydrogen bonding, and charged interactions as well as physical interactions such as mechanical interlocking.

Surface energy of a solid (dried) film is the reversible work required to create a unit surface area at a constant temperature and pressure, γₛ=δG/δA)_{T,P}. Solid surface energies are measured indirectly by probing with known liquids, using contact angle measurements. One instrument with which to make these measurements is a K12 tensiometer made by Kruss. Skin has surface energies around 38-39 milliNewtons/meter. Polymers or polymer mixtures/blends that have dry film surface energies in this range will typically give good adhesion to skin. Non-limiting examples are SANCURE 2710® (B. F. Goodrich, Cleveland, Ohio), a polyurethane which has a dry polymer film surface energy of 37.3 milliNewtons/meter, CARBOSET® 19X2 and CARBOSET® XL28 (both also available from B. F. Goodrich), acrylic polymers with γₛ=38.4 milliNewtons/meter and γₛ=37.9 milliNewtons/meter respectively, and poly(hydroxyethyl methacrylate) which has a γₛ=37 milliNewtons/meter. Generally, γₛ of about 20 to about 45 milliNewtons/meter will be preferred for adhesion to skin.

A full description of the surface energy of polymers and a listing of these values for many polymers can be found in the Polymer Handbook, Eds. J. Bandrup, E. H. Immergut, and E. A. Grulke, Wiley & Sons, 1999, 4^{th} Edition.

b. Young's (tensile) modulus: The ratio of the stress to the strain of a polymer in the elastic region is known as the Young's modulus or the tensile modulus. This term describes the stiffness or toughness of a polymer and therefore its ability to be flexible and move with the substrate onto which it is applied. Generally, the smaller the number, the more flexible the polymer and the larger the elongation to break. Generally, low density polyethylene with 165.5 MegaPascals, thermoplastic polyurethanes, 8 MegaPascals, and flexible poly(vinyl chloride) with 4.8-12.4 MegaPascals have preferred values of for tensile moduli. Examples of polymers with higher tensile modulus values are poly(methyl methacrylate) with 2400-3400 MegaPascals, rigid poly(vinyl chloride) with a value of 2800 MegaPascals, and polycarbonate with 2480 MegaPascals. For this invention, smaller values for Young's modulus are preferred.

For a listing of Young's modulus or tensile modulus values for many classes of polymers, see Polymer Handbook, Eds. J. Bandrup, E. H. Immergut, and E. A. Grulke, Wiley & Sons, 1999, 4^{th} Edition.

c. Glass transition temperature (T_{g}): The glass transition temperature is the critical temperature that separates glassy polymer behavior from flexible or “rubbery” polymer behavior. Above the T_{g}, molecular motions within a polymer (i.e., rubbery polymers) take place on the time scale of the experiment, below, they do not (i.e., glassy polymers). It also approximates the minimum temperature above which films are formed. As with Young's Modulus, the T_{g} relates to the brittleness or flexibility of the polymer film. The higher the T_{g}, the more brittle the film is and the larger the range of the glassy polymer; the smaller the value of T_{g}, the more flexible the film and the larger the range of the rubbery polymer.

For purposes of the present invention, a more flexible film is preferable because it will allow the film to move more easily with the skin. However, it is not necessary to only use a polymer or polymer mix with a low T_{g} to obtain a flexible film. It is also possible to obtain a flexible film either by using a polymer with a low T_{g}, such as poly(2-ethylhexyl acrylate) (T_{g}≈−50° C.), or poly(ethyl acrylate) (T_{g}≈−24° C.), or by using a polymer with a higher T_{g}, such as poly(methyl) methacrylate (T_{g}≈277° C.), in conjunction with a plasticizer, such as dibutyl phthalate or camphor, which has the function of decreasing the T_{g} of the film and making the film more easily deformable.

A full description of the glass transition temperature and a listing of the T_{g} for many polymers can be found in the Polymer Handbook, Eds. J. Bandrup, E. H. Immergut, and E. A. Grulke, Wiley & Sons, 1999, 4^{th} Edition.

d. Drying rate: A faster drying rate of the carrier composition is more preferred in this invention; a faster drying composition will allow less time prior to the application of sunscreen and therefore less preparation time before engaging in the activity for which sunscreen is required. The drying rate of the indicator will in part be dictated by the evaporation rate of the solvent and other diluents included in the carrier composition. The evaporation rate of any given solvent is a function of many parameters, such as vapor pressure of the liquid at the temperature under consideration, the latent heat of evaporation of the liquid, the humidity of the atmosphere, the surface tension of the liquid, as well as several other factors. With mixtures of solvents, the rate of evaporation is further dependent upon additional characteristics. Because of the many factors leading to evaporation rate, it is therefore very difficult to a priori predict which liquids are best used. A solvent's volatility is used as a guide to determine evaporation rate because vapor pressure is one of the prime factors governing the rate at which evaporation takes place. The boiling point (T_{b}) of the solvent is often used to estimate whether a solvent is more or less volatile. Roughly speaking, boiling points can be classified into three groups: low boilers (T_{b}<100° C.), medium boilers (100° C.<T_{b}<˜150° C.), and high boilers (T_{b}>150° C.). Generally, vapor pressure is inversely proportional to boiling point, i.e., lower boilers have higher vapor pressure (or higher volatility) at a given temperature.

More volatile solvents (solvents with a higher vapor pressure at a given temperature) will lead to carrier compositions that dry faster. In choosing a diluent, the more volatile solvents (those with lower boiling points) will be preferred in order to obtain a faster drying carrier composition. Non-limiting examples of preferred, faster drying, volatile solvents are ethyl acetate, acetone, heptane, ethanol, isopropyl acetate, isopropanol, methyl ethyl ketone, n-butyl acetate, n-propyl acetate, methyl acetate, isobutanol, n-propanol, n-butanol, n-amyl acetate, n-amyl alcohol, and isododecane.

The use of boiling points to classify volatility of solvents is only a guide for picking the appropriate solvent. Exceptions exists in which lower boiling points do not correspond to higher volatilities. One such example is found when comparing propyl alcohol (lower boiling point, slower evaporation rate) and propyl acetate (higher boiling point, faster evaporation rate). Therefore, liquids and solvents that do not have low boiling points are also useful in the compositions of this invention. For more information regarding boiling points and vapor pressure of liquids, see Solvents, T. H. Durrans, Chapman and Hall Ltd., 1971, 8^{th} Edition.

In a preferred embodiment of the invention, indicators are prepared by combining (e.g., by mixing) a carrier composition with a photochromic molecule. In this embodiment of the invention, a preferred indicator comprises:

1) the photochromic molecule described above, and

2) a carrier composition comprising

(a) a silicone gum,

(b) a preservative,

(c) a solvent,

(d) an organosiloxane resin,

(e) a gelling agent, and

(f) a stabilizer.

Ingredient (a) is a silicone gum. Suitable silicone gums are exemplified by those disclosed in PCT Patent Publication Nos. WO 97/17058 and WO 97/17059. Suitable silicone gums are exemplified by dimethicone gums and phenyl methyl silicone gums. Dimethicone gums are known in the art and commercially available as SE® 30 and SE® 63 from General Electric Company of Schenectady, N.Y. Phenyl methyl silicone gum is available as 88778, also from General Electric. Ingredient (a) typically comprises about 2% to about 20% of the carrier composition.

Ingredient (b) is a preservative, as described above. Ingredient (b) typically comprises 0.1 to % to about 1%.

Ingredient (c) is a volatile solvent such as ethyl acetate, alcohols such as ethanol, isopropanol, isobutanol, n-propanol, n-butanol, and n-amyl alcohol, dimethylsulfoxide, acetone, methyl ethyl ketone, isopropyl acetate, n-butyl acetate, n-propyl acetate, methyl acetate, n-amyl acetate, hydrocarbons such as heptane and isododecane, and combinations thereof. Preferably, the solvent is an alcohol or a hydrocarbon. Ingredient (c) comprises about 10% to about 90%, preferably about 15% to about 80%, more preferably about 20% to about 70% of the carrier composition.

Ingredient (d) is an organosiloxane resin. Suitable organosiloxane resins include the MQ resins discussed above, which are disclosed in International Patent Publication Numbers WO 97/17058 and WO 97/17059. Suitable organosiloxane resins include WACKER® 803 and 804 (available from Wacker Silicones Corporation of Adrian, Mich.) and GE® SR1000MQ and GE® 1170-002 (available from General Electric Company of Schenectady, N.Y.). Ingredient (d) typically comprises about 0.1% to about 40% of the carrier composition.

Ingredient (e) is a gelling agent. Suitable gelling agents are known in the art and are commercially available. Suitable gelling agents are available from Rheox, Inc., of Hightstown, N.J.; and these gelling agents include BENTONE® products, as described above. Ingredient (e) typically comprises about 0.1% to about 20% of the carrier composition.

Ingredient (f) is a stabilizer as described above. The stabilizer typically comprises from about 0.25% to about 3% of the carrier composition.

Although the amounts of carrier composition and photochromic molecule in the indicator will vary depending on the type of carrier composition and the photochromic molecule selected, the indicator will generally contain about 0.01% to about 10 weight % photochromic molecule and about 90% to about 99.99 weight % carrier composition. The resulting mixture can be put in a dispenser, such as an ink pen, felt marker, or stamp pad, or preformed-film, such as a temporary tattoo.

In an alternative embodiment of the invention, a dosimeter-type indicator can be prepared. The dosimeter-type indicator changes color once, e.g., to indicate maximum sun exposure for the day. The dosimeter-type indicator comprises:

i) an irreversible molecule as described above, and

ii) a carrier composition as described above.

This invention further relates to a method for preventing UV radiation overexposure to skin. The method comprises:

i. applying to the skin an indicator comprising a photochromic molecule and a carrier,

ii. applying a sunscreen over the indicator and the skin, and thereafter

iii. reapplying the sunscreen when the indicator changes color.

When the indicator is a pre-formed thin film, it is applied to the skin, for example, by pressing the film against the skin or wetting the film and thereafter pressing it against the skin.

When the indicator is a fluid film, the indicator is applied to the skin by any convenient means. For example, the indicator can be applied by drawing, using an ink indicator in a pen or marker, by stamping, using an ink indicator in a stamp pad, or by rubbing, using a balm stick. In a preferred embodiment of the invention, the fluid film indicator is allowed to dry, preferably for several minutes, after step i and before step ii.

Sunscreen may be applied and reapplied by any convenient means. However, the sunscreen must be applied over the indicator. Typically, the indicator changes color within a few seconds of exposure to UV radiation, and reverts to its original color within one minute after the UV radiation exposure ends.

### EXAMPLES

These examples are intended to illustrate the invention to those skilled in the art and should not be interpreted as limiting the scope of the invention set forth in the claims.

All percentages are by weight unless otherwise indicated. The sunscreen used in the examples is COPPERTONE® All Day Protection Sport SPF 48, available from Schering-Plough HealthCare Products, Inc. of Memphis, Tenn.

### Example 1

Part A is prepared by adding 43.58 grams GE(®) SR1000 MQ resin and 53.72 grams ethyl acetate to a glass jar and mixing by hand for 3 to 5 minutes.

Part B is prepared by dissolving 0.0585 grams of PHOTOSOL® 0265 from PPG in 7.50 grams ethyl acetate.

Part C is prepared by combining 156.20 grams SE 30 silicone gum and 141.40 grams ethyl acetate in a glass jar and mixing mechanically. Ethyl acetate is added to the jar first, and the silicone gum is added while stirring. Stirring speed is increased up to about 1200 RPM as the gum dissolves and viscosity increases. Total stirring time is about 5 hours.

Example 1. Solvent-Borne Polymer Film Indicator

| Component | Supplier | Percentage |
|---|---|---|
| Dimethicone silicone gum | GE (SE 30) | 5.54% |
| Organosiloxane resin | GE (SR 1000MQ) | 19.71% |
| Ethyl Acetate | Sigma-Aldrich | 61.02% |
| Photochromic Dye | PPG (Photosol Dye 0265) | 0.25% |
| Gelling agent | Rheox, Inc. (Bentone ISD) | 13.49% |

The indicator of Example 1 is prepared by combining 10.41 g of Part A, 3.19 g of Bentone ISD Gel, and 7.52 g of Part B in a glass jar and mixing at 1600 RPM for 18 minutes. Part C is then added (2.49 g) and mixed at 1600 RPM for 1 minute and then 1000 RPM for 30 minutes.

This formula is placed in a UC-18B Applicator Pen with a feather brush tip made by Mitsubishi Pencil Co. LTD. This is used for testing. The formula is applied on the skin and allowed a few minutes to dry. When long UV light (λ=365 nm) from a UV lamp (Spectroline ENF-240, 0.2 amps, 60 Hz) irradiates the sample, the spot turns to a very visible blue. Removing the UV light allows the indicator to revert back to its colorless state within 1 minute. However, if sunscreen (SPF 48) is applied over the indicator on the skin before removing it from the UV light, the blue color disappears. A second irradiation with the sunscreen applied over the indicator does not turn the indicator blue.

This formulation is fast drying, low in tack, and gives a dramatic color change.

Example 2. Water-Borne Polymer Film Indicator

| Component | Supplier | Percentage |
|---|---|---|
| Wet polyurethane polymer | BF Goodrich(Sancure 2710) | 25.0% |
| Ethanol | Sigma Aldrich | 72.5% |
| Ethyl Acetate | Sigma Aldrich | 2.3% |
| Photochromic Dye | PPG (Photosol Dye 0265) | 0.2% |

0.086 grams of Photosol Dye 0265 is dissolved in 2.018 grams of ethyl acetate. 2.5% (0.25 grams) of this dye solution is added to 7.25 grams of ethanol. 2.5 grams of Sancure 2710 (polyurethane polymer) is added and the mixture is mixed by hand for a few minutes, until a thick consistency is achieved. The dye appeared to be homogeneously dissolved/dispersed into the composition. The indicator is placed onto the skin, dries quickly (within approximately 1 minute), is colorless, and not tacky after drying. It is then exposed to UV light at 365 nm and is observed to change color. Removal of the UV light causes the indicator, now blue in color, to revert back to its colorless form.

Example 3. Water-Soluble Polymer Blend Film Indicator

| Component | Supplier (Product name) | Percentage |
|---|---|---|
| Wet polyurethane | BF Goodrich (Sancure 2710) | 22.5% |
| polymer | | |
| Solid polycellulose | Dow Chemical Co. (Methocel 40-202) | 1.0% |
| polymer | | |
| Ethanol | Sigma Aldrich | 72.5% |
| Ethyl Acetate | Sigma Aldrich | 2.3% |
| Photochromic Dye | PPG (Photosol Dye 0265) | 0.2% |
| Water | — | 1.5% |

0.086 grams of Photosol Dye 0265 is dissolved in 2.018 grams of ethyl acetate. 2.5% (0.25 grams) of this dye solution is added to 7.25 grams of ethanol. 0.15 grams of water is added, along with 2.25 grams of Sancure 2710 (polyurethane polymer), and 0.1 grams of Methocel 40-202 (hydroxypropyl methylcellulose). This is then mixed by hand for a few minutes, until a thick consistency is achieved. The dye appears to be homogeneously dissolved or dispersed into the film. The indicator is placed onto the skin, dries quickly (within approximately 1 minute), is colorless, and not tacky after drying. It is then exposed to UV light at 365 nm and is observed to change color. Removal of the UV light causes the indicator, now blue in color, to revert back to its colorless form.

Reference Example 1

Part A is prepared by adding 43.58 grams GE® SR1000 MQ resin and 53.72 grams ethyl acetate to a glass jar and mixing by hand for 3 to 5 minutes.

Part B is prepared by dissolving 0.0585 grams of a photochromic molecule.

Part C is prepared by combining 156.20 grams SE 30 silicone gum and 141.40 grams ethyl acetate in a glass jar and mixing mechanically. Ethyl acetate is added to the jar first, and the silicone gum is added while stirring. Stirring speed is increased up to about 1200 RPM as the gum dissolves and viscosity increases. Total stirring time is about 5 hours.

Solvent-Borne Polymer Film Indicator:

| Component | Supplier | Percentage |
|---|---|---|
| Dimethicone silicone gum | GE (SE 30) | 5.54% |
| Organosiloxane resin | GE (SR1000MQ) | 19.71% |
| Ethyl Acetate | Sigma-Aldrich | 61.02% |
| Photochromic Molecule | | 0.25% |
| Gelling agent | Rheox, Inc. (Bentone ISD) | 13.49% |

An indicator is prepared by combining 10.41 g of Part A, 3.19 g of Bentone ISD Gel, and 7.52 g of Part B in a glass jar and mixing at 1600 RPM for 18 minutes. Part C is then added (2.49 g) and mixed at 1600 RPM for 1 minute and then 1000 RPM for 30 minutes.

This indicator is placed in a UC-18B Applicator Pen with a feather brush tip made by Mitsubishi Pencil Co. LTD.

### Example 4

An indicator is prepared according to Reference Example 1. The photochromic molecule is 1,3,3-trimethyl spiro(indoline-2,3′-(3H)naphth(2,1-b)(1,4)oxazine).

The indicator is applied in a spot on the skin and allowed a few minutes to dry. When long UV light (λ=365 nm) from a UV lamp (Spectroline ENF-240, 0.2 amps, 60 Hz) irradiates the sample, the spot changes color. Removing the UV light allows the indicator to revert back to its original color. However, if sunscreen (SPF 48) is applied over the indicator on the skin before removing it from the UV light, the indicator changes back to its original color. A second irradiation with the sunscreen applied over the indicator does not change the color of the indicator.

### Example 5

An indicator is prepared according to Reference Example 1. The photochromic molecule is 1′,3′,3-trimethylspiro(2H-1)benzopyran-2,2′-indoline, 6-nitro-1′,3′,3′-trimethylbenzoindolinospiropyran.

The indicator is applied in a spot on the skin and allowed a few minutes to dry. When long UV light (λ=365 nm) from a UV lamp (Spectroline ENF-240, 0.2 amps, 60 Hz) irradiates the sample, the spot changes color. Removing the UV light allows the indicator to revert back to its original color. However, if sunscreen (SPF 48) is applied over the indicator on the skin before removing it from the UV light, the indicator changes back to its original color. A second irradiation with the sunscreen applied over the indicator does not change the color of the indicator.

### Example 6

An indicator is prepared according to Reference Example 1. The photochromic molecule is a fulgide having the structure

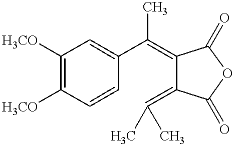

The indicator is applied in a spot on the skin and allowed a few minutes to dry. When long UV light (λ=365 nm) from a UV lamp (Spectroline ENF-240, 0.2 amps, 60 Hz) irradiates the sample, the spot changes color. Removing the UV light allows the indicator to revert back to its original color. However, if sunscreen (SPF 48) is applied over the indicator on the skin before removing it from the UV light, the indicator changes back to its original color. A second irradiation with the sunscreen applied over the indicator does not change the color of the indicator.

### Example 7

An indicator is prepared according to Reference Example 1. The photochromic molecule is fulgimide having the structure

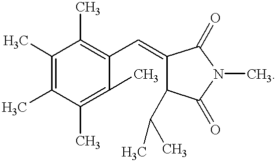

The indicator is applied in a spot on the skin and allowed a few minutes to dry. When long UV light (λ=365 nm) from a UV lamp (Spectroline ENF-240, 0.2 amps, 60 Hz) irradiates the sample, the spot changes color. Removing the UV light allows the indicator to revert back to its original color. However, if sunscreen (SPF 48) is applied over the indicator on the skin before removing it from the UV light, the indicator changes back to its original color. A second irradiation with the sunscreen applied over the indicator does not change the color of the indicator.

### Example 8

An indicator is prepared according to Reference Example 1. The photochromic molecule is a bisimidazole.

The indicator is applied in a spot on the skin and allowed a few minutes to dry. When long UV light (λ=365 nm) from a UV lamp (Spectroline ENF-240, 0.2 amps, 60 Hz) irradiates the sample, the spot changes color. Removing the UV light allows the indicator to revert back to its original color. However, if sunscreen (SPF 48) is applied over the indicator on the skin before removing it from the UV light, the indicator changes back to its original color. A second irradiation with the sunscreen applied over the indicator does not change the color of the indicator.

### Example 9

An indicator is prepared according to Reference Example 1. The photochromic molecule is a viologen derivative.

The indicator is applied in a spot on the skin and allowed a few minutes to dry. When long UV light (λ=365 nm) from a UV lamp (Spectroline ENF-240, 0.2 amps, 60 Hz) irradiates the sample, the spot changes color. Removing the UV light allows the indicator to revert back to its original color. However, if sunscreen (SPF 48) is applied over the indicator on the skin before removing it from the UV light, the indicator changes back to its original color. A second irradiation with the sunscreen applied over the indicator does not change the color of the indicator.

### Example 10

### Oil-based Stamper Indicator

Oil-based Stamper Ink:

| Component | Supplier | Percentage |
|---|---|---|
| Exxate 600 | Custom solvent from Dow Corning | 65.58% |
| Propylene Gylcol | Sigma-Aldrich | 29.90% |
| Butyl Ether | | |
| Polyvinylbutyral | Sigma-Aldrich | 4.18% |
| Photochromic | Color Change | 0.17% |
| Molecule, “Red 2” | | |
| N-pentacosane | Sigma-Aldrich | 0.17% |

A stamper ink is prepared by combining 30.93 grams of Exxate 600, 14.1 grams of Propylene Gylcol Butyl Ether, 0.07822 grams of n-pentacosane and 0.08205 grams of “Red 2” photochromic dye in a glass beaker and mixing for approximately 30 minutes until the dye dissolves. 1.97 grams of Polyvinylbutyral is then added to the solution to increase viscosity.

The stamper ink solution is used in combination with a common rubber stamper. The stamp can be applied to a piece of paper or the skin. The indicator is applied in a spot on the skin and allowed a few minutes to dry. When long UV light (λ=365 nm) from a UV lamp (Spectroline ENF-240, 0.2 amps, 60 Hz) irradiates the sample, the spot changes color. Removing the UV light allows the indicator to revert back to its original color.

### Example 11

### Temporary Tattoo Indicator

A temporary tattoo kit was purchased from HPS (Lake Dallas, Tex. 75065). The tattoo kit was originally designed for use with inkjets and other printing devices. In this example, the kit is used with the oil-based photochromic stamper ink described in example 10.

The temporary tattoo is prepared by stamping photochromic ink on to the shiny side of the tattoo paper. The tattoo paper is then coated with a thin layer of ‘tattoo tack’ and allowed to dry (slightly tacky after drying). The finished tattoo is then covered with a non-stick paper (wax paper) for protection.

A single tattoo is cut from a sheet of stamped tattoos and placed on the skin. The tattoo is held in place with a wet paper towel for thirty seconds to facilitate the transfer to the skin. The tattoo is initially colorless and cannot be seen other than a faint sheen which outlines the design. When long UV light (λ=365 nm) from a UV lamp (Spectroline ENF-240, 0.2 amps, 60 Hz) irradiates the tattoo, the spot changes color. Removing the UV light allows the tattoo to revert back to its original color. However, if sunscreen (SPF 48) is applied over the tattoo on the skin before removing it from the UV light, the indicator changes back to its original color. A second irradiation with the sunscreen applied over the indicator does not change the color of the indicator.

What is claimed is:

1. A sun protection kit comprising:
(A) a sunscreen, and
(B) an indicator comprising a photochromic molecule and a carrier, and optionally, (C) instructions for use of the kit.

2. The kit of claim 1, wherein the photochromic molecule is selected from the group consisting of spirooxazines, spiropyrans, fulgides, fulgimides, bisimidazoles, viologen derivatives, and combinations thereof.

3. The kit of claim 1, wherein the indicator undergoes a color change selected from the group consisting of:
1) the indicator is colorless in the absence of UV radiation and becomes colored when exposed to UV radiation,
2) the indicator is colored in the absence of UV radiation and becomes a different color when exposed to UV radiation,
3) the indicator is colored in the absence of UV radiation and becomes colorless when exposed to UV radiation.

4. An indicator comprising:
i) a photochromic molecule, and
ii) a carrier,
wherein the indicator can be applied directly to skin without a separate adhesive layer.

5. The indicator of claim 4, wherein the photochromic molecule is selected from the group consisting of spirooxazines, spiropyrans, fulgides, fulgimides, bisimidazoles, viologen derivatives, and combinations thereof.

6. The indicator of claim 5, wherein the spirooxazine has the formula: 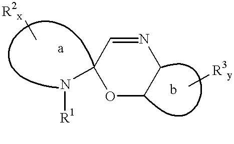
wherein group a is a spiro-linked group bonded to a carbon atom at the 3 position in the oxazine ring and group b is a fused group bonded to carbon atoms at the 5,6 position in the oxazine ring,
R¹ is selected from the group consisting of a hydrogen atom and a lower monovalent hydrocarbon group,
each R² is independently selected from the group consisting of a hydrogen atom, and a substituent bonded to a carbon atom in the ring of group a,
each R³ is independently selected from the group consisting of a hydrogen atom and a substituent bonded to a carbon atom in the ring of group b,
x represents a number of hydrogen atoms and substituents bonded to carbon atoms in the ring of group a, and
y represents a number of hydrogen atoms and substituents bonded to carbon atoms in the ring of group b.

7. The indicator of claim 5, wherein the spiropyran has the formula: 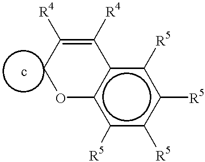
wherein group c is a spiro-linked group selected from the group consisting of heterocyclic, substituted heterocyclic groups, heteroaromatic groups, and substituted heteroaromatic groups,
each R⁴ is independently selected from the group consisting of a hydrogen atom, monovalent hydrocarbon groups, substituted monovalent hydrocarbon groups, heterogeneous groups, substituted heterogeneous groups, aromatic groups, substituted aromatic groups, heteroaromatic groups, substituted heteroaromatic groups, carbocyclic groups, substituted carbocyclic groups, heterocyclic groups, substituted heterocyclic groups, nitro groups, hydroxy groups, and amino groups, and
each R⁵ is independently selected from the group consisting of R⁴, with the proviso that two instances of R⁵ may be bonded together to form a fused ring selected from the group consisting of aromatic groups, substituted aromatic groups, heteroaromatic groups, substituted heteroaromatic groups, cyclic groups, substituted cyclic groups, heterocyclic groups, and substituted heterocyclic groups.

8. The indicator of claim 5, wherein the fulgide has the formula: 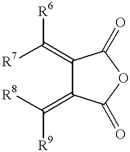
wherein R⁶, R⁸ and R⁹ are each independently selected from the group consisting of a hydrogen atom, a monovalent hydrocarbon group, a substituted monovalent hydrocarbon group, a heterogeneous group, and a substituted heterogeneous group, and
R⁷ is selected from the group consisting of an aromatic group, a substituted aromatic group, a heteroaromatic group, a substituted heteroaromatic group, a carbocyclic group, a substituted carbocyclic group, a heterocyclic group, and a substituted heterocyclic group.

9. The indicator of claim 5, wherein the fulgimide has the formula: 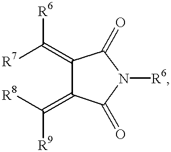
wherein R⁶, R⁸ and R⁹ are each independently selected from the group consisting of a hydrogen atom, a monovalent hydrocarbon group, a substituted monovalent hydrocarbon group, a heterogeneous group, and a substituted heterogeneous group; and
R⁷ is selected from the group consisting of an aromatic group, a substituted aromatic group, a heteroaromatic group, a substituted heteroaromatic group, a carbocyclic group, a substituted carbocyclic group, a heterocyclic group, and a substituted heterocyclic group.

10. The indicator of claim 5, wherein the bisimidazole has the formula: 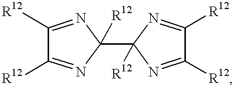
wherein each R¹² is independently selected from the group consisting of an aromatic group, a substituted aromatic group, a heteroaromatic group, and a substituted heteroaromatic group.

11. The indicator of claim 5, wherein the viologen derivative has the formula: 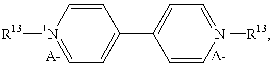
wherein each R¹³ is independently selected from the group consisting of a monovalent hydrocarbon group, a substituted monovalent hydrocarbon group, a heterogeneous group, a substituted heterogeneous group, an aromatic group, a substituted aromatic group, a heteroaromatic group, a substituted heteroaromatic group, a carbocyclic group, a substituted carbocyclic group, a heterocyclic group, and a substituted heterocyclic group; and each A represents a halogen atom.

12. The indicator of claim 4, wherein the carrier is essentially UV transparent.

13. The indicator of claim 4, wherein the indicator has a form selected from the group consisting of a solid film and a fluid film.

14. The indicator of claim 13, wherein the solid film is a temporary tattoo.

15. The indicator of claim 13, wherein the solid film comprises a portion colored with a design that is augmented by development of the photochromic molecule.

16. The indicator of claim 13, wherein the solid film comprises areas with differing concentrations of the photochromic molecule.

17. The indicator of claim 13, wherein the photochromic molecule is printed in areas in the film adjacent to colored reference zones.

18. The indicator of claim 13, wherein the fluid film is in a form selected from the group consisting of a balm stick, an ink pen, a marker, a stamp-pad with stamper, a lotion, and combinations thereof.

19. The indicator of claim 5, wherein the carrier is a carrier composition.

20. The indicator of claim 19, wherein the carrier composition comprises a (i) film-forming polymer, (ii) a liquid diluent, and (iii) one or more optional components.

21. The indicator of claim 20, wherein the optional component (iii) is selected from the group consisting of coalescents, coloring agents, plasticizers, preservatives, resins, gelling agents, stabilizers, chelating agents, dispersing agents, fillers, thickeners, wax additives, prophylactic agents, wetting agents, and combinations thereof.

22. An indicator comprising:
i) a photochromic molecule, and
ii) a carrier composition,
wherein the carrier composition has a surface energy of about 20 milliNewtons/meter to about 45 milliNewtons/meter.

23. A dosimeter-type indicator comprising
i) a molecule selected from the group consisting of an irreversible molecule and a combination of a photochromic molecule and an irreversible molecule, and
ii) a carrier composition, wherein the indicator can be applied directly to skin without a separate adhesive layer.

24. The indicator of claim 23, wherein the irreversible molecule is selected from the group consisting of 18-molybdodiphosphoric acid, oxazolidine-dione compounds, xanthenone compounds, tetrazolium salts, photosensitive onium salts and dyes which change color when protonated, 2(2′,4′dinitrobenzy)pyridine, and combinations thereof.

25. A method for preventing ultraviolet radiation overexposure to skin comprising:
i. applying to the skin an indicator comprising a photochromic molecule and a carrier,
ii. applying a sunscreen over the indicator and the skin, and thereafter
iii. reapplying the sunscreen when the indicator changes color.
